(19) United States
(12) Patent Application Publication
(10) Pub. No.: US 2024/0203019 A1
(43) Pub. Date: Jun. 20, 2024

(54) METHOD AND MODEL FOR THREE-DIMENSIONAL CHARACTERIZATION OF MOLYBDENUM DISULFIDE SAMPLE BASED ON MACHINE LEARNING, AND USE THEREOF

(71) Applicant: JIANGSU UNIVERSITY, Zhenjiang (CN)

(72) Inventors: Quan WANG, Zhenjiang (CN); Yue HE, Zhenjiang (CN); Yi JU, Zhenjiang (CN)

(73) Assignee: JIANGSU UNIVERSITY, Zhenjiang (CN)

(21) Appl. No.: 17/798,067

(22) PCT Filed: Sep. 26, 2021
(86) PCT No.: PCT/CN2021/120532

§ 371 Date: Aug. 5, 2022

(30) Foreign Application Priority Data
Apr. 27, 2021 (CN) 202110457805.9

(51) Int. Cl. G06T 15/00; G01Q 60/24; G06T 5/20; G06T 5/70; G06T 7/11; G06V 10/25; G06V 10/56

(52) U.S. Cl. CPC G06T 15/00; G01Q 60/24; G06T 5/20; G06T 5/70; G06T 7/11; G06V 10/25; G06V 10/56

(57) ABSTRACT

A method for three-dimensional (3D) characterization of a molybdenum disulfide sample based on machine learning (ML) includes: subjecting the molybdenum disulfide sample to optical imaging and atomic force microscopy (AFM) characterization; constructing and training a model through a random forest (RF) algorithm based on a dataset of a correspondence between a color feature of an optical image and AFM height data of the molybdenum disulfide sample; and inputting a color feature value of the optical image of the molybdenum disulfide sample into the model to acquire height data of the molybdenum disulfide sample, and filtering to remove a local noise and a local abnormal point, so as to acquire a final 3D characterization image. The present disclosure has high characterization accuracy and is helpful for scientific researchers to quickly analyze the thickness of a molybdenum disulfide sample through optical imaging without AFM or other characterization instrumentation.

### TECHNICAL FIELD

The present disclosure belongs to the technical field of two-dimensional (2D) material detection, and relates to a method and model for three-dimensional (3D) characterization of a molybdenum disulfide sample based on machine learning (ML), and a use thereof.

### BACKGROUND

With the continuous emergence of two-dimensional materials, the excellent mechanical, electrical and optical properties of 2D materials have attracted widespread attention. Among the 2D materials, molybdenum disulfide is an important raw material for lubricants and has diamagnetic properties. Molybdenum disulfide has an energy bandgap of 1.8 eV, and a single-layer molybdenum disulfide transistor has an electron mobility up to about 500 cm²/(V·s) and an on-off current ratio up to 1×10⁸. Therefore, molybdenum disulfide can be widely used in the field of nanotransistors. Single-layer molybdenum disulfide grown by chemical vapor deposition (CVD) is prone to introduce an impurity, thereby affecting device performance. In contrast, naturally occurring molybdenum disulfide in its bulk form has a uniform texture, and single-layer molybdenum disulfide prepared by a micromechanical exfoliation method has more excellent properties than that prepared by the CVD method. Therefore, the micromechanical exfoliation method can provide researchers with test samples with more excellent properties. However, since light is reflected at the boundary layers between molybdenum disulfide and silicon dioxide and between silicon dioxide and silicon and interferes with each other, molybdenum disulfide with different thicknesses shows different colors in the optical image. In recent years, with the maturity of the machine learning (ML) technology, great breakthroughs have been made in the k-nearest neighbors (KNN) algorithm, the random forest (RF) algorithm and other ML algorithms. However, although ML has been gradually applied in various industries, its use in the field of 2D materials is still insufficient due to the lack of suitable feature extraction methods.

### SUMMARY

The present disclosure provides a method and a model for three-dimensional (3D) characterization of a molybdenum disulfide sample based on machine learning (ML), and a use thereof. The present disclosure performs the 3D characterization of the molybdenum disulfide sample through optical imaging, and achieves the higher characterization accuracy.

The present disclosure achieves the above technical objective through the following technical means.

A method for 3D characterization of a molybdenum disulfide sample based on ML includes the following steps:
(1) optical image acquisition: preparing the molybdenum disulfide sample, and acquiring an optical image of the molybdenum disulfide sample through a microscope;
(2) image processing: subjecting the optical image acquired in the step (1) to denoising and mean filtering;
(3) atomic force microscopy (AFM) characterization: performing the AFM characterization in a same local region for the optical image acquisition to acquire AFM height data of the local region of the molybdenum disulfide sample;
(4) region of interest (ROI) segmentation: segmenting a local region, corresponding to an ROI indicated by an AFM characterization result acquired in the step (3), in the optical image processed in the step (2);
(5) image feature extraction and dataset establishment: extracting a color feature dataset of the local region segmented in the optical image; using the AFM height data as a target dataset; and combining each pixel datum in the color feature dataset of the optical image with a respective pixel datum in the target dataset of the AFM height data to form a feature dataset of a height image of the molybdenum disulfide sample;
(6) dataset splitting and ML model training: splitting the feature dataset into a training set and a testing set, wherein the training set is used for training a model, and the testing set is used for validating an accuracy of the model; constructing the model by a random forest (RF) algorithm based on the training set, and training the model by controlling a number of random trees based on the testing set, so as to improve the accuracy of the model; and finally exporting the model;
(7) new image import operation: processing a target molybdenum disulfide sample according to the step (1) and the step (2) to obtain an optical image, extracting a color feature value of the optical image, bringing the color feature value into the model acquired in the step (6), and calculating height data of the target molybdenum disulfide sample; and
(8) 3D image filtering: filtering a 3D image acquired in the step (7) to remove a local noise and a local abnormal point, so as to acquire a final 3D characterization image.

Further, the optical image is acquired by the microscope under a linearly adjustable light source, and one optical image is acquired per 0.25 mm² area of the molybdenum disulfide sample.

Further, in the ROI segmentation in the step (4), an ROI of the optical image is segmented, scaled to a same pixel size as an AFM image, and further segmented.

Further, in the image feature extraction in the step (4), an effect of a light intensity of the segmented ROI on a color is reduced by

$$[ \frac{{A}_{1} ( L ) * B}{{L}_{silicon}} , \frac{{A}_{2} ( L ) * G}{{L}_{silicon}} , \frac{{A}_{3} ( L ) * R}{{L}_{silicon}} ] ,$$

where L denotes a light intensity depth; A(L) denotes an optical compensation function; B, G, and R denote color feature values; and L_{silicon} denotes a light intensity depth of a silicon wafer region.

Further, in the step (5), an effect of a characterization accuracy error of the AFM height data on the accuracy of the model after the training is reduced by

$$H = \{ \begin{matrix}0 , & {h}_{n} < 0 \\ 2 \times [ \frac{{h}_{n}}{2} ] , & {h}_{n} \geq 0\end{matrix} ,$$

where H denotes a processed height dataset; and hₙ denotes an n-th original height data.

Further, in the dataset splitting in the step (6), a split ratio of the training set and the testing set is 4:1.

Further, in the 3D image filtering in the step (8), the height data are subjected to mean filtering by a 3*3 mask; and in the step (4), the segmented local region of the optical image has a pixel value of 500*500 pt.

The present disclosure further provides a model for 3D characterization of a molybdenum disulfide sample, which is constructed by the method for 3D characterization of the molybdenum disulfide sample based on ML.

The present disclosure further provides a use of the model for 3D characterization of the molybdenum disulfide sample in the 3D characterization of the molybdenum disulfide sample based on an optical image of the molybdenum disulfide sample.

The present disclosure has the following beneficial effects.

The present disclosure combines two-dimensional materials and the ML technology to perform 3D characterization of a molybdenum disulfide sample through optical imaging, and achieves the higher characterization accuracy. The present disclosure is helpful for scientific researchers to quickly analyze the thickness of a molybdenum disulfide sample through optical imaging without AFM or other characterization instrumentation. The present disclosure also makes a preliminary exploration on the method of 3D characterization of samples through optical imaging for scientific researchers in future.

### BRIEF DESCRIPTION OF THE DRAWINGS

FIG. 1 is a flowchart of a method for three-dimensional (3D) characterization of a molybdenum disulfide sample based on machine learning (ML) according to the present disclosure.

FIG. 2 is an image of the molybdenum disulfide sample under an optical microscope.

FIG. 3 shows the analysis of a 3D morphology of the molybdenum disulfide sample by atomic force microscopy (AFM).

FIG. 4 is a 3D image acquired by the method for 3D characterization of the molybdenum disulfide sample according to the present disclosure.

### DETAILED DESCRIPTION OF THE EMBODIMENTS

The present disclosure is described in further detail below with reference to the drawings and specific embodiments. It should be understood that these embodiments are only used to illustrate the present disclosure, rather than to limit the scope of the present disclosure. Those skilled in the art should understand that any equivalent modifications made to the present disclosure should fall within the scope defined by the claims of the present disclosure.

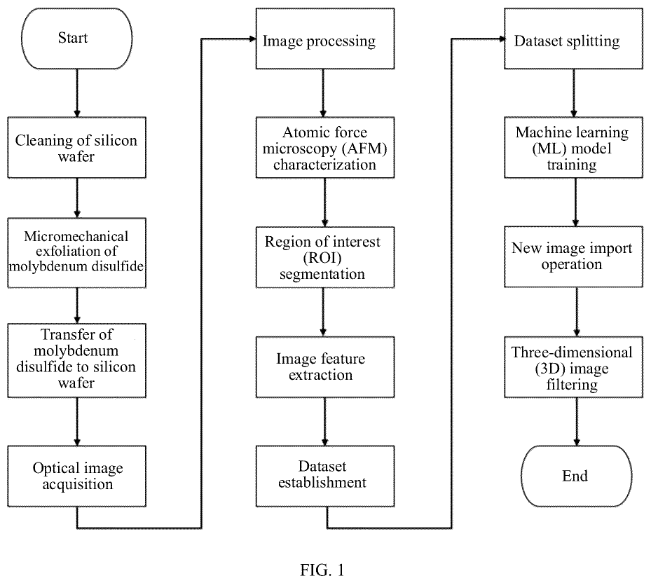

As shown in FIG. 1, the present disclosure provides a method for three-dimensional (3D) characterization of a molybdenum disulfide sample based on machine learning (ML). It mainly includes the following steps: optical image acquisition, image processing, atomic force microscopy (AFM) characterization, region of interest (ROI) segmentation, image feature extraction, dataset establishment, dataset splitting, ML model training, new image import operation, and 3D image filtering.

An optical image is acquired by a microscope. The molybdenum disulfide sample is prepared by a micromechanical exfoliation method using a 1*1 cm, 300 nm heavily doped P-type silicon oxide wafer as a substrate. The substrate is first heated in acetone for 10 min and ultrasonically cleaned for 10 min, and then ultrasonically cleaned by isoethanol for 5 min. Residual acetone is then removed. The substrate is rinsed with deionized water and blown dry with nitrogen gas, such that a surface of the substrate is clean. The molybdenum disulfide sample is prepared by a micromechanical exfoliation method and a bulk molybdenum disulfide sample is taken by a Nitto tape, and the sample is sufficiently thinned by tearing it 3-6 times. The tape with the molybdenum disulfide sample is picked up by using tweezers, and the sample is pressed against the cleaned silicon oxide wafer with a finger, and air bubbles therebetween are squeezed out to make the molybdenum disulfide sample fully adhere to the silicon oxide wafer. Finally, the tape is removed to acquire the final molybdenum disulfide sample. The optical image is acquired under a linearly adjustable light source, and one optical image is acquired per 0.25 mm² area of the molybdenum disulfide sample. The acquired optical image is subjected to denoising and mean filtering.

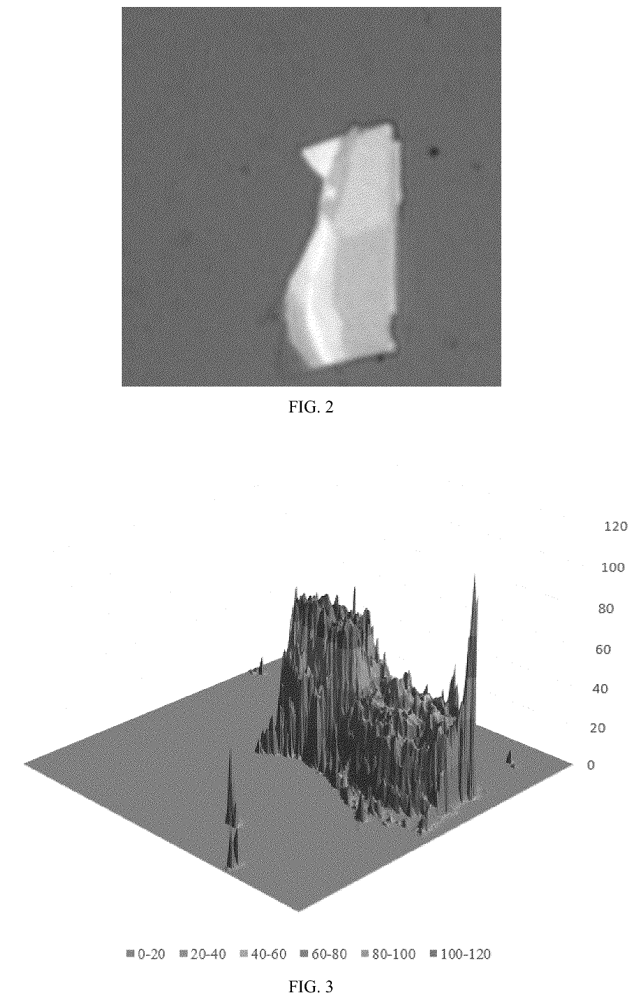

AFM characterization is performed in the same local region for the optical image acquisition to acquire AFM height data of the local region of the molybdenum disulfide sample, as shown in FIG. 3. Then, a local region, corresponding to an ROI indicated by an AFM characterization result, is segmented in the denoised and filtered optical image, thereby completing the ROI segmentation. A color feature dataset of the local region segmented in the optical image is extracted, the AFM height data are used as a target dataset, and each pixel datum in the color feature dataset of the optical image is combined with a respective pixel datum in the target dataset of the AFM height data to form a feature dataset of a height image of the molybdenum disulfide sample, thereby completing image feature extraction and dataset establishment.

Specifically, in the image feature extraction, an effect of a light intensity of the segmented ROI on a color is reduced by

$$[ \frac{{A}_{1} ( L ) * B}{{L}_{silicon}} , \frac{{A}_{2} ( L ) * G}{{L}_{silicon}} , \frac{{A}_{3} ( L ) * R}{{L}_{silicon}} ] ,$$

where L denotes a light intensity depth; A(L) denotes an optical compensation function; B, G, and R denote color feature values; and L_{silicon} denotes a light intensity depth of a silicon wafer region. Through such processing, a final color feature value of the molybdenum disulfide sample is acquired. An effect of a characterization accuracy error of the AFM height data on the accuracy of the model after the training is reduced by

$$H = \{ \begin{matrix}0 , & {h}_{n} < 0 \\ 2 \times [ \frac{{h}_{n}}{2} ] , & {h}_{n} \geq 0\end{matrix} ,$$

where H denotes a processed height dataset; and hₙ denotes an n-th original height data.

Then, feature dataset splitting and ML model training are conducted. The feature dataset is split into a training set and a testing set, and the training set is used for training a model, and the testing set is used for validating an accuracy of the model. A split ratio of the training set and the testing set is 4:1.

The model is constructed by a random forest (RF) algorithm based on the training set, and trained by controlling a number of random trees based on the testing set, so as to improve the accuracy of the model. Finally, the model is exported.

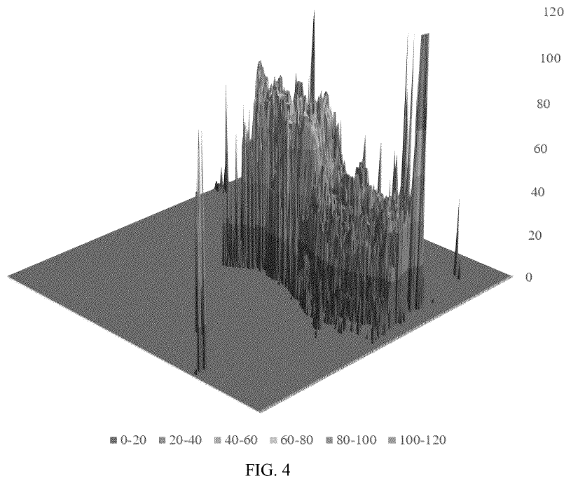

The steps of optical image acquisition and image processing are performed on a target molybdenum disulfide sample to obtain an optical image. A color feature value of the optical image of the sample is extracted, and brought into the exported model to calculate height data of the target molybdenum disulfide sample. An acquired 3D image is filtered by a 3*3 mask to remove a local noise and a local abnormal point, so as to acquire a final 3D characterization image, as shown in FIG. 4.

The above embodiments are preferred implementations of the present disclosure, but the present disclosure is not limited to the above implementations. Any obvious improvement, substitution, or modification made by those skilled in the art without departing from the essence of the present disclosure should fall within the protection scope of the present disclosure.

What is claimed is:

1. A method for a three-dimensional characterization of a molybdenum disulfide sample based on a machine learning, comprising the following steps:
(1) an optical image acquisition: preparing the molybdenum disulfide sample, and acquiring an optical image of the molybdenum disulfide sample through a microscope;
(2) an image processing: subjecting the optical image acquired in the step (1) to a denoising and a mean filtering;
(3) an atomic force microscopy (AFM) characterization: performing the AFM characterization in a same local region for the optical image acquisition to acquire an AFM height data of a first local region of the molybdenum disulfide sample;
(4) a region of interest (ROI) segmentation: segmenting a second local region, corresponding to an ROI indicated by an AFM characterization result acquired in the step (3), in the optical image processed in the step (2);
(5) an image feature extraction and a dataset establishment: extracting a color feature dataset of the second local region segmented in the optical image; using the AFM height data as a target dataset; and combining each pixel datum in the color feature dataset of the optical image with a respective pixel datum in the target dataset of the AFM height data to form a feature dataset of a height image of the molybdenum disulfide sample;
(6) a dataset splitting and a machine learning model training: splitting the feature dataset into a training set and a testing set, wherein the training set is used for training a model, and the testing set is used for validating an accuracy of the model; constructing the model by a random forest algorithm based on the training set, and training the model by controlling a number of random trees based on the testing set to improve the accuracy of the model; and finally exporting the model;
(7) a new image import operation: processing a target molybdenum disulfide sample according to the step (1) and the step (2) to obtain the optical image, extracting a color feature value of the optical image, bringing the color feature value into the model acquired in the step (6), and calculating a height data of the target molybdenum disulfide sample; and
(8) a three-dimensional image filtering: filtering a three-dimensional image acquired in the step (7) to remove a local noise and a local abnormal point to acquire a final three-dimensional characterization image.

2. The method for the three-dimensional characterization of the molybdenum disulfide sample based on the machine learning according to claim 1, wherein the optical image is acquired by the microscope under a linearly adjustable light source, and one optical image is acquired per 0.25 mm² area of the molybdenum disulfide sample.

3. The method for the three-dimensional characterization of the molybdenum disulfide sample based on the machine learning according to claim 1, wherein in the ROI segmentation in the step (4), the ROI of the optical image is segmented, scaled to a same pixel size as an AFM image, and further segmented.

4. The method for the three-dimensional characterization of the molybdenum disulfide sample based on the machine learning according to claim 1, wherein in the image feature extraction in the step (5), an effect of a light intensity of a segmented ROI on a color is reduced by
$$[ \frac{{A}_{1} ( L ) * B}{{L}_{silicon}} , \frac{{A}_{2} ( L ) * G}{{L}_{silicon}} , \frac{{A}_{3} ( L ) * R}{{L}_{silicon}} ] ,$$
wherein L denotes a light intensity depth; A(L) denotes an optical compensation function; B, G, and R denote color feature values; and L_{silicon} denotes a light intensity depth of a silicon wafer region.

5. The method for the three-dimensional characterization of the molybdenum disulfide sample based on the machine learning according to claim 1, wherein in the step (5), an effect of a characterization accuracy error of the AFM height data on the accuracy of the model after the machine learning model training is reduced by
$$H = \{ \begin{matrix}0 , & {h}_{n} < 0 \\ 2 \times [ \frac{{h}_{n}}{2} ] , & {h}_{n} \geq 0\end{matrix} ,$$
wherein H denotes a processed height dataset; and hₙ denotes an n-th original height data.

6. The method for the three-dimensional characterization of the molybdenum disulfide sample based on the machine learning according to claim 1, wherein in the dataset splitting in the step (6), a split ratio of the training set and the testing set is 4:1.

7. The method for the three-dimensional characterization of the molybdenum disulfide sample based on the machine learning according to claim 1, wherein in the three-dimensional image filtering in the step (8), the height data are subjected to the mean filtering by a 3*3 mask.

8. The method for the three-dimensional characterization of the molybdenum disulfide sample based on the machine learning according to claim 1, wherein in the step (4), the second local region of the optical image has a pixel value of 500*500 pt.

9. A model for the three-dimensional characterization of the molybdenum disulfide sample constructed by the method for the three-dimensional characterization of the molybdenum disulfide sample based on the machine learning according to claim 1.

10. A use of the model for the three-dimensional characterization of the molybdenum disulfide sample according to claim 9 in the three-dimensional characterization of the molybdenum disulfide sample based on the optical image of the molybdenum disulfide sample.

11. The model for the three-dimensional characterization of the molybdenum disulfide sample constructed by the method for the three-dimensional characterization of the molybdenum disulfide sample based on the machine learning according to claim 9, wherein the optical image is acquired by the microscope under a linearly adjustable light source, and one optical image is acquired per 0.25 mm² area of the molybdenum disulfide sample.

12. The model for the three-dimensional characterization of the molybdenum disulfide sample constructed by the method for the three-dimensional characterization of the molybdenum disulfide sample based on the machine learning according to claim 9, wherein in the ROI segmentation in the step (4), the ROI of the optical image is segmented, scaled to a same pixel size as an AFM image, and further segmented.

13. The model for the three-dimensional characterization of the molybdenum disulfide sample constructed by the method for three-dimensional characterization of the molybdenum disulfide sample based on the machine learning according to claim 9, wherein in the image feature extraction in the step (5), an effect of a light intensity of a segmented ROI on a color is reduced by
$$[ \frac{{A}_{1} ( L ) * B}{{L}_{silicon}} , \frac{{A}_{2} ( L ) * G}{{L}_{silicon}} , \frac{{A}_{3} ( L ) * R}{{L}_{silicon}} ] ,$$
wherein L denotes a light intensity depth; A(L) denotes an optical compensation function; B, G, and R denote color feature values; and L_{silicon} denotes a light intensity depth of a silicon wafer region.

14. The model for the three-dimensional characterization of the molybdenum disulfide sample constructed by the method for three-dimensional characterization of the molybdenum disulfide sample based on the machine learning according to claim 9, wherein in the step (5), an effect of a characterization accuracy error of the AFM height data on the accuracy of the model after the machine learning model training is reduced by
$$H = \{ \begin{matrix}0 , & {h}_{n} < 0 \\ 2 \times [ \frac{{h}_{n}}{2} ] , & {h}_{n} \geq 0\end{matrix} ,$$
wherein H denotes a processed height dataset; and h, denotes an n-th original height data.

15. The model for the three-dimensional characterization of the molybdenum disulfide sample constructed by the method for three-dimensional characterization of the molybdenum disulfide sample based on the machine learning according to claim 9, wherein in the dataset splitting in the step (6), a split ratio of the training set and the testing set is 4:1.

16. The model for the three-dimensional characterization of the molybdenum disulfide sample constructed by the method for three-dimensional characterization of the molybdenum disulfide sample based on the machine learning according to claim 9, wherein in the three-dimensional image filtering in the step (8), the height data are subjected to the mean filtering by a 3*3 mask.

17. The model for the three-dimensional characterization of the molybdenum disulfide sample constructed by the method for three-dimensional characterization of the molybdenum disulfide sample based on the machine learning according to claim 9, wherein in the step (4), the second local region of the optical image has a pixel value of 500*500 pt.
